(19) United States
(12) Patent Application Publication
(10) Pub. No.: US 2018/0318026 A1
(43) Pub. Date: Nov. 8, 2018

(54) DISPOSABLE SHEATH FOR ULTRASOUND PROBE MOUNTED ON REUSABLE NEEDLE STRUCTURE

(71) Applicant: Gynesonics, Inc., Redwood City, CA (US)

(72) Inventor: Brian Placek, Manhattan, KS (US)

(73) Assignee: Gynesonics, Inc., Redwood City, CA (US)

(21) Appl. No.: 16/037,548

(22) Filed: Jul. 17, 2018

Related U.S. Application Data
(63) Continuation of application No. PCT/US17/14753, filed on Jan. 24, 2017.
(60) Provisional application No. 62/287,818, filed on Jan. 27, 2016.

(51) Int. Cl. A61B 46/17; A61B 8/12; A61B 8/00; A61B 18/14

(52) U.S. Cl. CPC A61B 46/17; A61B 8/12; A61B 2018/00577; A61B 18/1477; A61B 8/445

(57) ABSTRACT

A disposable sheath is provided for a radiofrequency ablation device which includes both an ultrasonic imaging probe and a detachable needle advancement assembly. The sheath includes a distal region which conforms closely to a shaft of the ultrasonic imaging probe but drapes more loosely over a handle of the ultrasonic imaging probe. The disposable sheath allows attachment features on both the ultrasonic imaging probe and the needle advancement assembly to be easily visualized and easily connected together.

### CROSS REFERENCE TO RELATED APPLICATIONS

This application is a continuation of PCT Application No. PCT/US17/14753 (Attorney Docket No. 31992-715.601), filed Jan. 24, 2017, which claims priority to U.S. Provisional Patent Application Ser. No. 62/287,818, filed Jan. 27, 2016, the entire content of which are incorporated herein by reference.

### BACKGROUND OF THE INVENTION

### 1. Field of the Invention

The present invention relates generally to medical methods and apparatus. More particularly, the present invention relates to sheaths and methods of use with reusable ultrasonic imaging probes that are removably attached to needle deployment assemblies for the treatment of uterine fibroids and other tissues.

Current medical treatments of organs and tissues within a patient's body often use a needle or other body for delivery of energy, therapeutic agents or the like. Optionally, the methods use ultrasound imaging to observe and identify a treatment target and track the position of the needle relative to the treatment target.

Of particular interest to the present invention, a treatment for uterine fibroids has recently been proposed which relies on transvaginal or laparoscopic positioning of a treatment device in the patient's uterus. A radiofrequency or other energy or therapeutic delivery needle is deployed from the device into the fibroid, and energy and/or therapeutic substances are delivered in order to ablate or treat the fibroid. To facilitate locating the fibroids and positioning the needles within the fibroids, the device includes an ultrasonic imaging array with an adjustable field of view in a generally forward or lateral direction relative to an axial shaft which carries the needle. The needle is advanced from the shaft and across the field of view so that the needle can be visualized and directed into the tissue and the targeted fibroid.

In specific systems as described, for example, in U.S. Patent Publication 2014/0073910, an ultrasound imaging probe is removably attached to a needle advancement assembly so that the relatively expensive ultrasound probe can be sterilized for reuse and the less expensive needle advancement assembly can be disposed. Sterilization of the ultrasound probe before reuse is essential, but the presence of sensitive components, such as the ultrasounds transducer, can make such sterilization difficult.

To at least partially overcome the challenges of sterilizing ultrasound probes and other surgical implements, sheaths can be placed over the probes to prevent or inhibit contamination in the first place. Sheaths are often used with endoscopes and other imaging probes, and it has been proposed to use sheaths on ultrasound imaging components that are combined with needles and other interventional tools, as mentioned in the prior patents and published applications listed below.

While the concept of using sheaths for isolating ultrasound probes is well known, none of the presently proposed approaches are completely suited for combined ultrasound imaging probes and needle advancement assemblies of the type described in U.S. Patent Publication 2014/0073910, commonly owned with the present application, the full disclosure of which is incorporated herein by reference. In particular, such assemblies require precise alignment of the ultrasound probe and the needle advancement assembly since the projected needle paths will be calculated based on such alignment. The sheaths must also provide close conformance over the transducer to exclude air while allowing the ultrasound transducer to pivot relative to a fixed portion of the shaft. Pivoting an ultrasound transducer would tear and/or displace many or most prior art sheaths which are not well fitted to the underlying. Due to the ill-fitting sheaths of the prior art, users often have to resort to using rubber bands, tape, or other such informal measures to in an attempt to improve performance. The probe assemblies of U.S. Patent Publication 2014/0073910 also require that the handle of the imaging component be covered but be sufficiently accessible to allow levers and other components on the covered handle to be manipulated during use, e.g. to deflect the imaging transducer during the procedure.

For these reasons, it would be desirable to provide improved sheaths and methods for sheathing reusable ultrasonic or other imaging components which may be combined with disposable needles or other energy components to form probes used for ablating tissues or performing other therapeutic or diagnostic protocols in tissue. It would be particularly useful to provide sheaths which are capable of conforming closely to an ultrasound transducer of the imaging component while not interfering with assembling the imaging component and the needle or other therapeutic component. The sheath should also allow any necessary manipulations of the ultrasound probe and in particular the probe handle. Additionally, it would be desirable if the position and settings of the probe were detectable with the sheath in place over the imaging components of the probe. At least some of these objectives will be met by the inventions described hereinafter.

### 2. Description of the Background Art

Placement of a sheath over the ultrasound component of a combination needle-ultrasound device is described in U.S. Patent Publication Nos. 20110022034, 2011/0218444, 2011/0028847, 20100081920, 2009/0171218, and 20080045793, and U.S. Pat. No. 6,368,280. Other patents of interest include U.S. Pat. Nos. 6,261,234 and 5,469,853. U.S. Patent Publication 2014/0073910 describes needle deployment systems having removable, reusable ultrasound probes of the type that can be used with the sheaths of the present invention.

### BRIEF SUMMARY OF THE INVENTION

The present invention provides disposable sheaths and methods for placing the sheaths over ultrasound probes which form a part of an ultrasound needle assembly for treating uterine fibroids or other tissue structure. The sheaths and methods are particularly useful since they have been configured to conform closely to the ultrasound imaging component of the probe in order to maximize the image clarity by eliminating air and other gaps which would degrade the image and decrease acoustic efficiency. While the sheath fits closely over the ultrasound transducer, it allows the transducer to pivot in order to be oriented properly for imaging and treatment without substantial displacement of the sheath which continues to closely adhere to the transducer. The sheath is also specially configured to allow attachment of the separate needle advancement component with the ultrasound probe after the probe has been placed in the sheath. In particular, as described below, the sheath allows a user to feel (or in some cases see) the outlines of one or more attachment features on the ultrasound probe which can then be mated with corresponding features on the needle advancement assembly, allowing precise alignment of the needle advancement component with the ultrasound probe component. While the sheath will normally conform to most or all of the length of a shaft of the imaging probe component, the sheath will be relatively loosely held over a handle portion of the imaging probe component. Such a loose fit allows the user to actuate levers and other components on handle in order to manipulate the imaging probe during use. While the sheath may be relatively loose over the handle portion of the probe, it still provides excellent isolation and reduction of contamination during the surgical procedure.

In a first aspect of the present invention, a disposable sheath is provided for use in combination with a radiofrequency ablation device. The radiofrequency ablation device includes (i) a reusable probe shaft and handle having an ultrasound imaging transducer near a distal end thereof and (ii) a disposable needle advancement assembly which is configured to be removably attached to a side of the probe shaft and handle. The sheath comprises a tubular body having a sealed distal end and an open proximal end. The tubular body is formed from a thin material and has a conforming distal portion which conforms closely to the distal end of the reusable probe shaft. In particular, the conforming distal portion of the tubular body of the sheath will fit tightly over at least the ultrasound imaging transducer of the probe shaft so that no air gaps remain over the transducer when the sheath is in place on the probe shaft. In contrast to the conforming distal portion of the tubular body of the sheath, a proximal portion of the sheath is configured to be loosely received over a proximal length of the probe shaft, typically being disposed over a handle portion attached to a proximal end of the probe shaft. Such a loose fit allows the user to manipulate levers and other components on the probe, typically on the probe handle, in order to manipulate the transducer or other component near the distal end of the probe shaft. The sheath will be particularly configured so that it does not interfere with attachment of the needle advancement assembly to the probe shaft when the sheath is in place over the probe shaft. In specific embodiments, a circumferential dimension of the tubular body of the sheath varies along its length so that (1) a circumferential dimension of a distal portion of the sheath matches a circumferential dimension around a distal portion probe shaft and (2) a circumferential dimension of a proximal portion of the sheath is greater than a circumferential dimension over a proximal portion of the probe shaft.

The tubular body of the disposable sheath will typically be formed from a thin polymer membrane, typically having a thickness in the range from 25 μm to 600 μm. The polymer may be any conventional polymer used for such medical sheaths, but will typically be a very thin, strong polymer which resists tearing and accidental penetration, such as a polyisoprene, polyurethane, latex, or the like. The sheath will usually be formed from a single material, but could be formed from more than one material, including being formed (i) as a laminate of two or more polymer layers or of polymer and non-polymer layers, (ii) from different polymers which are joined in different axial sections, (iii) being reinforced with strands or a mesh of the same or different polymers, or the like. For example, the distal conforming portion of the tubular body of the sheath may be formed from a particular elastic or other material, while the more proximal portions of the tubular body of the sheath could be formed from one or other materials as the performance characteristics of the proximal end are less challenging. The sheath may be formed by any conventional fabrication technology, such as dip-molding, casting, compression molding, liquid injection molding, and the like.

In specific embodiments, the portion of the sheath which covers the transducer may be sufficiently elastic to both closely conform to the shape of the transducer while allowing the transducer to pivot without substantially displacing the sheath. In other embodiments, the sheath may be formed from a less elastic material, but in those cases the sheath will usually be pre-shaped so that it still closely conforms to the geometry of at least the distal region of the probe shaft including the transducer and attachment regions, as discussed in more detail below. In instances where the shape and circumference of the probe shaft vary along the shaft length, it is necessary only that the circumference of the tubular body be similarly varied along its length so that it can conform to the specific profile of the probe shaft once the sheath has been placed there over.

In particular embodiments of the disposable sheaths of the present invention, the sheaths will be configured to closely conform to external attachment features on the shaft of the ultrasonic probe. The external attachment features are intended to engage or mate with corresponding features on the needle advancement assembly, so that the features may be joined and the ultrasonic probe and needle advancement assembly secured together so they can operate as one instrument. In such cases, the sheath will be configured so that, once placed over the probe shaft, the attachment features on the probe shaft remain detectable by shape (the user can feel the protrusions of the features) to allow a user to align the attachment features on the advancement assembly with the external attachment features on the probe shaft in order to allow precisely aligned attachment. Usually, the conforming distal portion of the disposable sheath will extend at its distal end over the ultrasound transducer and continue in a proximal direction so that it also covers at least some of the attachment features on the shaft. In many embodiments, the conforming distal portion of disposable sheath will extend over most or all of the length of the shaft portion of the ultrasound probe and will make a transition to the loose fitting region of the sheath only when it begins to cover the handle or other enlarged portion of the ultrasound probe.

In particular embodiments, the sheaths of the present invention will be configured to conform to an axial groove along one side of the probe shaft when an elongate body of the needle advancement assembly is received in the groove. In such embodiments, the external attachment features on the probe sheath may be located on either or preferably both sides of the groove.

In a second aspect of the present invention, a disposable sheath for use with a radiofrequency ablation device as described above comprises a tubular body having a sealed distal end and an open proximal end. The ultrasonic probe has external attachment features which engage attachment features on the needle advancement assembly, and the tubular body of the sheath is configured to conform to the external attachment features on the probe shaft so that the features are detectable through the sheath (tactilely and/or visible if the sheath is transparent) to allow the user to align the attachment features on the needle advancement assembly with those on the probe shaft prior to attachment.

The disposable sheath will have the dimensions and characteristics generally as described above with the first aspect of the present invention.

In addition, the disposable sheath of the second aspect may be formed to have a conforming distal portion which conforms closely to the distal end of the reusable probe shaft including at least the ultrasound imaging transducer. In such instances, no air gaps will remain over the transducer when the sheath is in place on the probe shaft, improving acoustic efficiency and image accuracy. A proximal portion of the sheath is loose and does not conform to a proximal length of the probe shaft, and the sheath will not interfere with attachment of the needle advancement assembly when the sheath is in place over the probe shaft.

The conforming distal portion of the disposable sheath of the second aspect may optionally extend proximally over at least some of the attachment features of the shaft, and the probe shaft will typically have an axial groove along one side thereof which receives the body of the needle attachment assembly. In such instances, the disposable sheath will lie between the elongate member of the needle advancement assembly and the wall of the groove in which the elongate member is received. Usually, attachment features will be formed on one or both sides of the groove in order to hold the needle advancement assembly in place within the groove.

In a third aspect of the present invention, a method for using an interventional surgical device having a reusable probe shaft with an ultrasound imaging transducer and a disposable needle advancement assembly comprises placing a disposable sheath over the probe shaft so that a distal portion of the sheath conforms closely to a distal region of the reusable probe shaft. The sheath will cover at least the ultrasound imaging transducer so that nowhere gaps remain over the transducer when the sheath is in place on the probe shaft. The needle advancement assembly is then attached to the probe shaft so that the sheath is positioned between the needle advancement assembly and the probe shaft over at least the entire length of the probe shaft.

Usually, the distal portion of the sheath further conforms closely to one or more attachment features on the distal portion of the probe shaft. Typically, shape of one or more of the attachment features is assumed by the sheath so that the attachment feature is apparent to the user by touch and or visually in the case of transparent sheaths. The user is thus able to align one or more attachment features on the needle advancement assembly with the one or more attachment features on the probe which are discernable through the sheath before attachment the needle advancement assembly to the probe shaft.

In most cases, a proximal portion of the sheath remains loosely draped over a proximal region of the probe, typically including the probe handle so that the user may continue to operate levers of the mechanisms on the handle or other distal region of the ultrasonic probe during performance of the imaging and treatment procedure.

### BRIEF DESCRIPTION OF THE DRAWINGS

FIG. 1 is a schematic illustration of a system that can use the sheath of the present invention. The system comprises a system controller, an image display, and a treatment probe having a deployable needle structure and imaging transducer. The sheath is illustrated in later figures.

FIG. 2 is a perspective view of a treatment assembly including and ultrasound probe component and a detachable needle advancement component. The treatment assembly is shown without the disposable sheath of the present invention.

FIG. 3 is a view of the treatment assembly of FIG. 2 illustrating the imaging component separated from the needle advancement component with portions broken away and portions enlarged.

FIG. 3A illustrates a distal end of the needle advancement component being connected to a distal end of the imaging component

FIG. 4 illustrates the ultrasound probe of FIGS. 1 through 3 together with two embodiments of the disposable sheath of the present invention.

FIG. 5 is a detailed view of the distal ends of the needle advancement assembly and the ultrasound imaging probe of the systems of FIGS. 1-3. The distal region of the ultrasound imaging probe is shown without the disposable sheath in place.

FIG. 6 is view similar to that of FIG. 5, except that the distal portion of the ultrasound image probe is shown with the sheath of the present invention in place prior to attachment of the needle advancement assembly.

FIG. 7 illustrates the fully assembled ultrasound treatment assembly with the present invention with the ultrasound imaging probe being covered by the disposable sheath of the present invention and the needle advancement assembly being attached but uncovered by the disposable sheath.

### DETAILED DESCRIPTION OF THE INVENTION

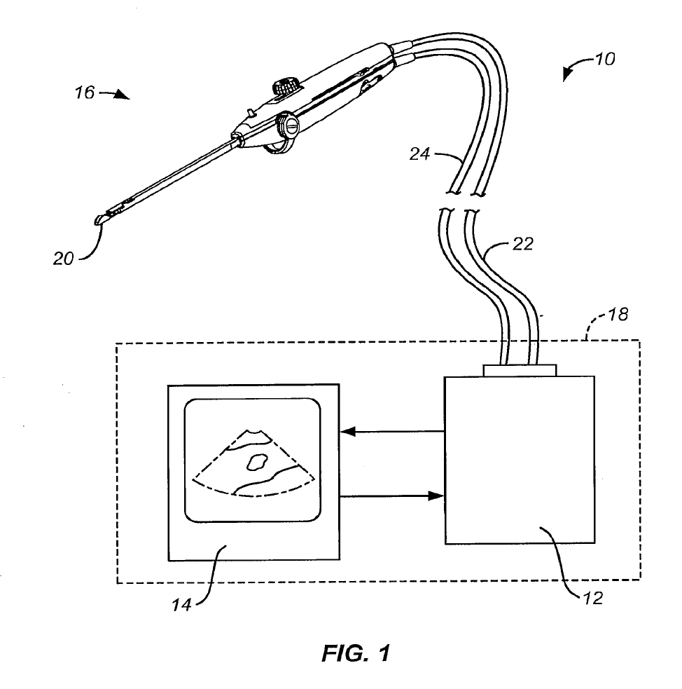

As illustrated in FIG. 1, a system 10 constructed in accordance with the principles of the present invention includes a system controller 12, an imaging display 14, and a treatment probe 16. The system controller 12 will typically be a microprocessor-based controller which allows both treatment parameters and imaging parameters to be set in a conventional manner. The display 14 will usually be included in a common enclosure 18 together with the controller 12, but could be provided in a separate enclosure. The treatment probe 16 includes an imaging transducer 20 which is connected to the controller 12 by an imaging cord 24. The controller 12 supplies power to the treatment probe via a treatment cord 22. The controller 12 will typically further include an interface for the treating physician to input information to the controller, such as a keyboard, touch screen, control panel or the like. Optionally, a touch panel may be part of the imaging display 14. The energy delivered to the treatment probe by the controller may be radiofrequency (RF) energy, microwave energy, a treatment plasma, heat, cold (cryogenic therapy), or any other conventional energy-mediated treatment modality. Alternatively or additionally, the treatment probe could be adapted to (1) deliver drugs or other therapeutic agents to the tissue anatomy to be treated, (2) perform tissue biopsy using a disposable needle removably attached to the ultrasound probe, or the like. In some embodiments, probe 16 plugs into an ultrasound system and into a separate radiofrequency generator or controller.

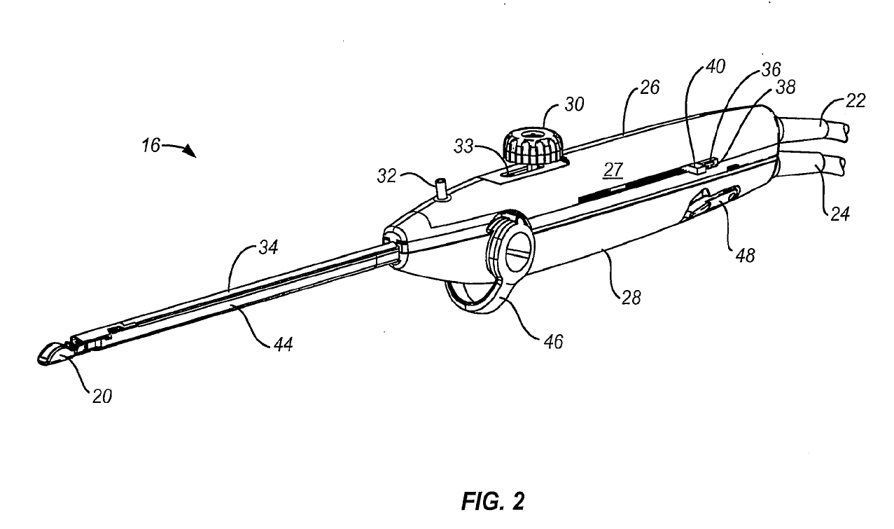

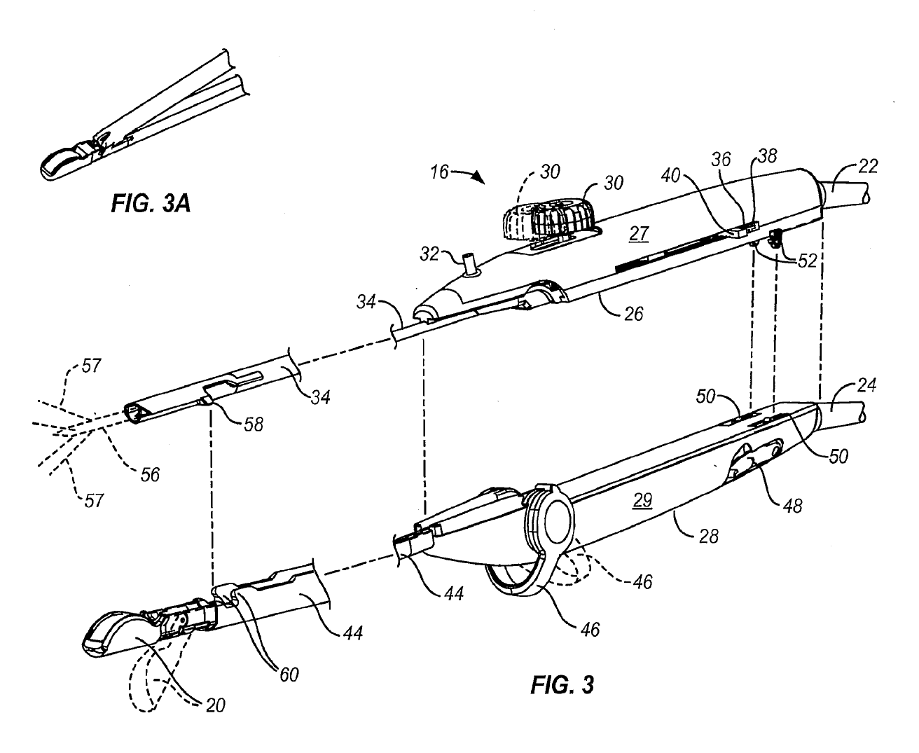

Referring now to FIGS. 2 and 3, the treatment probe 16 comprises a needle component 26 and an imaging component 28. The needle component and imaging component are constructed as separate units or assemblies which may be removably attached to each other for use. After use, the needle component may be separated and will typically be discarded while the imaging component will be sterilized for reuse. The treatment probe 16 is shown in its fully assembled configuration in FIG. 2 and is shown in its disassembled configuration in FIG. 3.

The needle component 26 comprises a handle portion 27 having a slidably mounted targeting knob 30 on its upper surface. The targeting knob 30 controls the positioning of internal stops within the handle which are monitored by the controller 12 (FIG. 1) in order to calculate the size and position of the boundaries of the targeting region and/or the safety region which are shown on the display 14. The stops will also serve to physically limit deployment of the needle 56 and optionally tines 57, as will be described in more detail below.

The needle 56 is deployed from the needle shaft 34, and the needle and optional tines together form a needle structure which may be constructed, for example, as previously described in commonly owned U.S. Pat. Nos. 8,206,300 and 8,262,574, the full disclosures of which are incorporated herein by reference.

The handle portion 27 of the needle component 26 further includes a fluid injection port 32 which allows saline or other fluids to be injected through the needle shaft 34 into a target region in the tissue being treated, such as the uterus. The needle handle 27 also includes a needle slide 36, a needle release 38, and a tine slide 40 which are used to deploy the needle 56 and tines 57, as will be described in more detail below. The imaging cord 24 is attachable at a proximal end of the handle portion 27 of the imaging component 28 for connection to the controller 12, as previously described.

The imaging component 28 comprises a handle portion 29 and an imaging shaft 44. A deflection lever 46 on the handle portion 29 can be retracted in order to downwardly deflect the imaging transducer 20, as shown in broken line in FIG. 3. A needle component release lever 48 is coupled to a pair of latches 50 which engage attachment features, e.g. hooks 52 on a bottom surface of the handle portion 27 of the needle component 26. The needle component 26 may be releasably attached to the imaging component 28 by first capturing a pair of attachment features, e.g. wings 58 (only one of which is shown in FIG. 3) on the needle shaft 34 beneath attachment features, e.g. hooks 60 on the imaging shaft 44, as shown in FIG. 3A. A bottom surface of the needle handle portion 27 may then be brought down over an upper surface of the imaging handle portion 29 so that the attachment features, e.g. hooks 52 engage the latches 50 to form a complete assembly of the treatment probe 16, where the handle portions together form a complete handle, for use in a procedure. After use, the needle component release lever 48 may be pulled in order to release the attachment features, e.g. hooks 52 from the latches 50, allowing the handle portions 27 and 29 to be separated.

In use, as will be described in more detail below, the targeting knob 30 is used to both position (translate) and adjust the size of a virtual treatment region which is projected onto the display 14 of the system 10. The knob 30 may be moved distally and proximally in a slot 33 on an upper surface of the handle portion 27 in order to translate the position of the treatment/safety region on the image, and the knob may also be rotated in order to adjust the size of the boundary of the treatment/safety region. Sliding and rotating the knob 30 will also adjust the position of mechanical stops in the handle portion 27 which limit the deployment of the needle 56 and tines 57 so that, once the virtual boundaries of the treatment/safety region have been selected on the real-time image, the needle and tines may be automatically advanced to the corresponding deployment positions by moving the needle slide 36 and tine slide 40 until their movement is arrested by the stops. The position of the treatment/safety region is also dependent on the location at which the physician holds the treatment probe 16 within the target tissue. Thus, advancement of the needle and tines using the slides 36 and 40 will result in the proper placement of the needle and tines within the target tissue only if the treatment probe position is held steady from the time the stops are set until advancement of the needle/tines is completed. In preferred embodiments, rotating the knob 30 will also determine the length of and/or power delivery during a treatment protocol. Thus, the knob may be used to virtually size the treatment/safety region based not only on the degree to which the tines have been advanced, but also the amount of energy which is being delivered to the target tissue, as described in U.S. Pat. No. 8,992,427, the full disclosure of which is incorporated herein by reference.

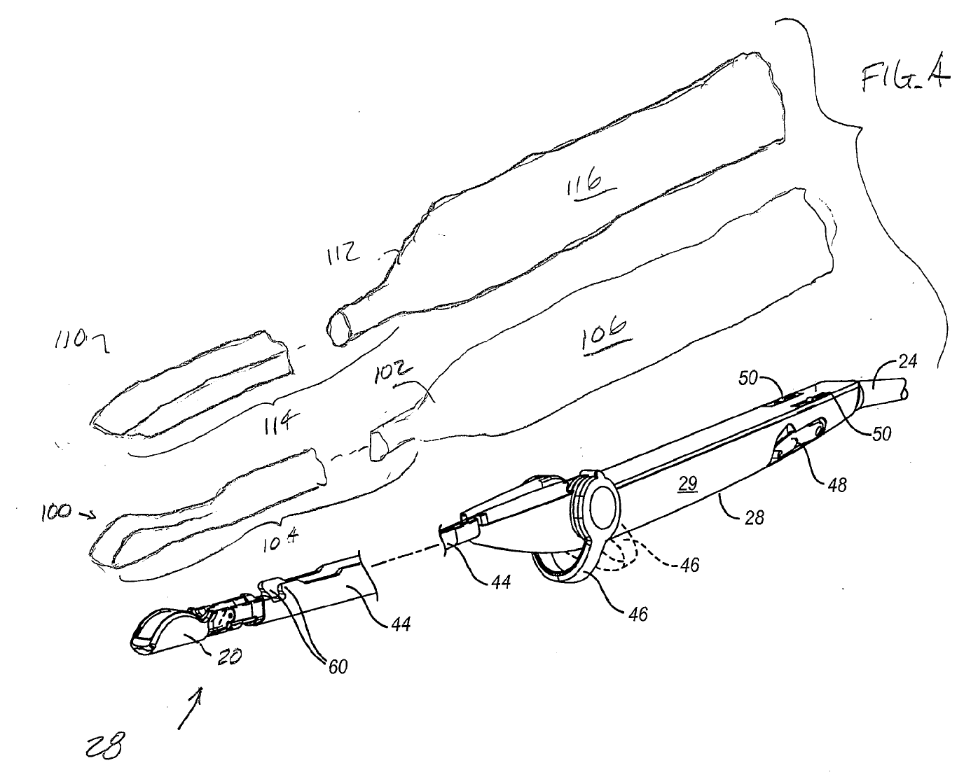

Referring now to FIG. 4, the imaging probe or component 28 is shown together with two embodiments of the disposable sheath of the present invention. A first disposable sheath embodiment 100 comprises a tubular body 102 having a closely conforming distal portion 104 formed of a thin polyisoprene or similar polymer material and a loosely fitting proximal portion 106 formed of a polyurethane, polyethylene or other similar polymer or polymer blend. The disposable sheath 100 is formed so that it has a shape or geometry which matches the features of the imaging probe or component 28 and highly elastic to allow its intrinsic shape to be placed over and removed from the probe or component 28.

Disposable sheath 110 also comprises a tubular body 112 having a conforming distal portion 114 and a loosely fitting proximal portion 116. The geometry of the conforming distal portion 114, however, does not necessarily match that of the distal portion of the shaft of the imaging probe 28. As that is the case, the controlling distal portion 114 of disposable sheath 110 will usually be undersized but also highly elastic so that it may be tightly conformed over the shaft of the imaging probe. In a third embodiment (not illustrated), the distal portion (equivalent to 104 or 114) may be non-conforming, loose, non-elastic, and likely clear.

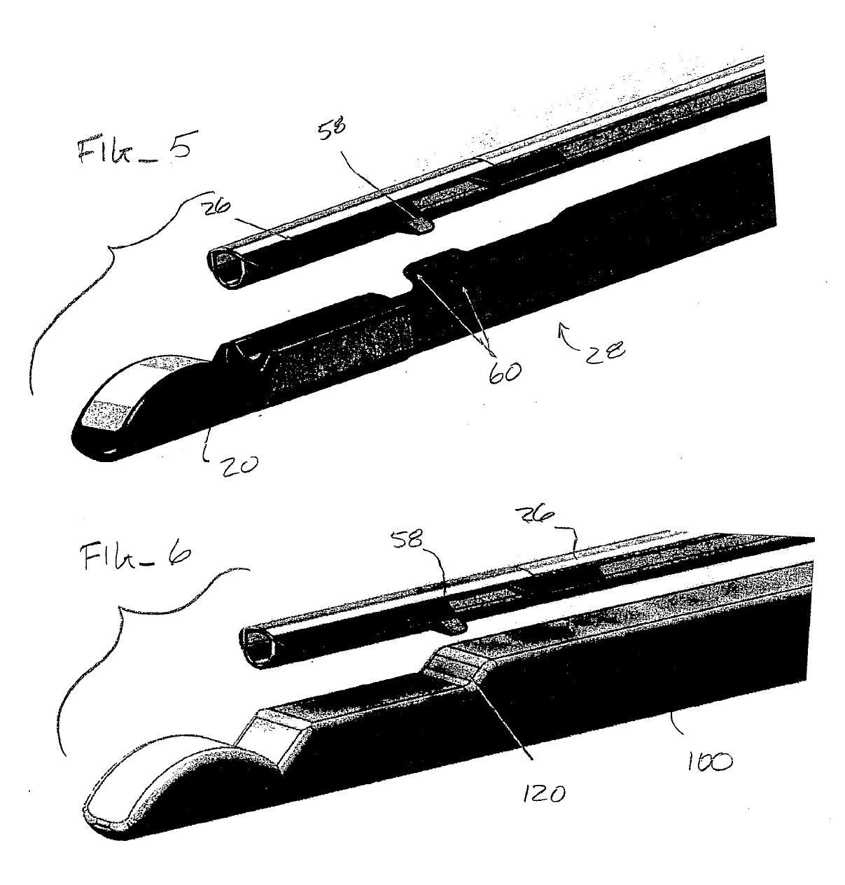

Referring now to FIG. 5, detailed views of the distal ends of the imaging probe 28 and needle advancement assembly 26 are shown. In particular, the attachment features, e.g. hooks 60, of the imaging probe 28 are shown adjacent the attachment features, e.g. wings 58, of the needle advancement assembly 26, but without the sheath covering the shaft of the imaging probe.

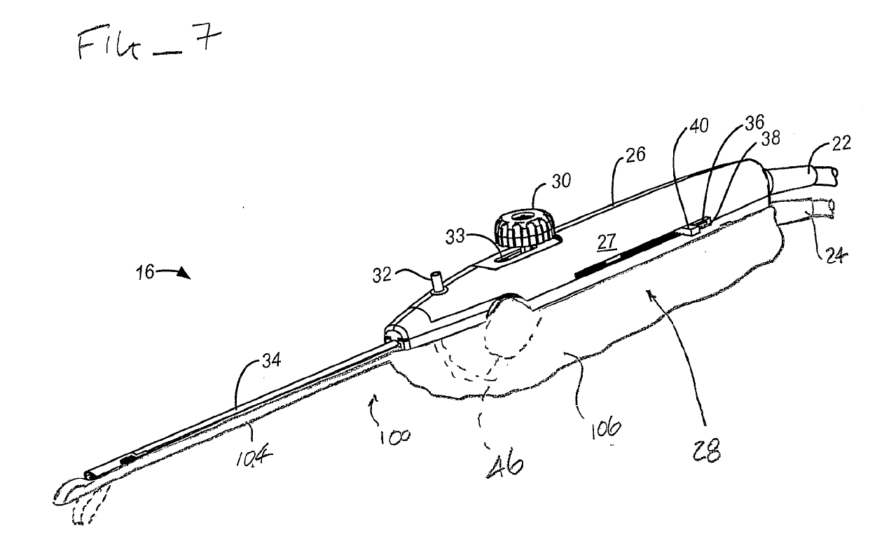

FIG. 6 is similar to FIG. 5, but shows the disposable sheath 100 in place over the distal shaft region of the imaging probe 28. In particular, it shows that the conforming distal portion 104 of the tubular body 102 conforms to the shape of the imaging probe and in particular provides a discernable edge or bump 120 where the attachment features 60 (hidden in FIG. 6) are located in beneath the sheath. The user is thus able to see the edge 120 and align the attachment features 58 on the needle advancement assembly 26 in order to facilitate attachment of the probe and advancement assembly, as show in FIG. 6. The probe cover assumes external geometry of the underlying imaging probe 28 so that a user can visually observe the location of underlying attachment features on the probe even if the sheath is opaque so that the probe itself is not directly visible.

In FIG. 7, it can be seen that the entire length of the shaft of imaging probe 28 is covered by the tightly conforming distal portion 104 of the disposable sheath 100. In contrast, the proximal portion 106 of the sheath is very loosely draped over the underlying handle 29 (hidden in FIG. 7) of the imaging probe, allowing the user to grasp and manipulate features on the handle, such as the transducer deflection lever 46 which is covered by the sheath but not constricted to prevent manipulation.

As shown in FIG. 7, the assembly of both the imaging probe 28 and the needle advancement assembly 26 is ready for use in a patient with the reusable imaging probe being fully isolated by the sheath to reduce or eliminate the need for sterilization after use. In contrast, the needle advancement assembly 26 may be fully exposed as it is disposable and not intended for reuse.

While preferred embodiments of the present invention have been shown and described herein; it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A disposable sheath for use in combination with a radiofrequency ablation device which includes (i) a reusable probe shaft having an ultrasound imaging transducer near a distal end thereof and (ii) a disposable needle advancement assembly which is configured to removably attach to a side of the probe shaft, wherein said disposable sheath comprises:
a tubular body having a sealed distal end and an open proximal end;
wherein the tubular body is formed from a thin material and has a distal portion configured to conform closely to the distal end of the reusable probe shaft including at least the ultrasound imaging transducer so that no air gaps remain over the transducer when the sheath is in place on the probe shaft;
wherein a proximal portion of the sheath is loose and does not conform to a proximal length of the probe shaft; and
wherein the sheath does not interfere with attachment of the needle advancement assembly when the sheath is in place over the probe shaft.

2. A disposable sheath as in claim 1, wherein the tubular body comprises a polymer membrane having a thickness in the range from 25 μm to 600 μm.

3. A disposable sheath as in claim 2, wherein the polymer is selected from the group consisting of polyisoprene, polyurethane, and latex.

4. A disposable sheath as in claim 3, wherein the sheath is formed by dip-molding, casting, compression molding, or liquid injection molding.

5. A disposable sheath as in claim 1, wherein at least a portion of the sheath which covers the transducer is sufficiently elastic to allow the transducer to pivot which the sheath remains closely conformed over the transducer.

6. A disposable sheath as in claim 1, wherein a circumferential dimension of the tubular body of the sheath varies along its length so that (1) a circumferential dimension of a distal portion of the sheath matches a circumferential dimension around a distal portion probe shaft and (2) a circumferential dimension of a proximal portion of the sheath is greater than a circumferential dimension over a proximal portion of the probe shaft.

7. A disposable sheath as in claim 1, wherein the probe has external attachment features which engage attachment features on the needle advancement assembly and wherein the tubular body is configured to conform to the external advancement features of the probe shaft so that the features are visible to allow a user to align the attachment features on the needle advancement assembly with the external attachment feature on the probe shaft prior to attachment.

8. A disposable sheath as in claim 7, wherein the conforming distal portion of the disposable sheath extends proximally over at least some of the attachment features on the shaft.

9. A disposable sheath as in claim 8, wherein the probe shaft has an axial groove along one side thereof which receives an elongate body of the needle advancement assembly.

10. A disposable sheath as in claim 9, wherein attachment features are formed on each side of the groove.

11. A disposable sheath for use in combination with a radiofrequency ablation device which includes (i) a reusable probe shaft having an ultrasound imaging transducer near a distal end thereof and (ii) a disposable needle advancement assembly which is configured to removably attach to a side of the probe shaft, wherein said disposable sheath comprises:
a tubular body having a scaled distal end and an open proximal end;
wherein the probe has external attachment features which engage attachment features on the needle advancement assembly and wherein the tubular body is configured to conform to the external advancement features of the probe shaft so that the features are visible to allow a user to align the attachment features on the needle advancement assembly with the external attachment feature on the probe shaft prior to attachment.

12. A disposable sheath as in claim 11, wherein the tubular body comprises a polymer membrane having a thickness in the range from 25 μm to 600 μm.

13. A disposable sheath as in claim 12, wherein the polymer is selected from the group consisting of polyisoprene, polyurethane, and latex.

14. A disposable sheath as in claim 13, where in the sheath is formed by dip-molding, casting, compression molding, or liquid injection molding.

15. A disposable sheath as in claim 11, wherein at least a portion of the sheath which covers the transducer is sufficiently elastic to allow the transducer to pivot which the sheath remains closely conformed over the transducer.

16. A disposable sheath as in claim 11, wherein a circumferential dimension of the tubular body of the sheath varies along its length so that (1) a circumferential dimension of a distal portion of the sheath matches a circumferential dimension around a distal portion probe shaft and (2) a circumferential dimension of a proximal portion of the sheath is greater than a circumferential dimension over a proximal portion of the probe shaft.

17. A disposable sheath as in claim 11, wherein the tubular body is formed from a thin material has a conforming distal portion which conforms closely to the distal end of the reusable probe shaft including at least the ultrasound imaging transducer so that no air gaps remain over the transducer when the sheath is in place on the probe shaft;
wherein a proximal portion of the sheath is loose and does not conform to a proximal length of the probe shaft; and
wherein the sheath does not interfere with attachment of the needle advancement assembly when the sheath is in place over the probe shaft.

18. A disposable sheath as in claim 17, wherein the conforming distal portion of the disposable sheath extends proximally over at least some of the attachment features on the shaft.

19. A disposable sheath as in claim 18, wherein the probe shaft has an axial groove along one side thereof which receives an elongate body of the needle advancement assembly.

20. A disposable sheath as in claim 19, wherein attachment features are formed on each side of the groove.

21. A method for using an interventional surgical device including (i) a reusable probe shaft having an ultrasound imaging transducer near a distal end thereof and (ii) a disposable needle advancement assembly which is configured to removably attach to a side of the probe shaft, said method comprising
placing a disposable sheath over the probe shaft so that a distal portion of the sheath which conforms closely to a distal region of the reusable probe shaft including at least the ultrasound imaging transducer so that no air gaps remain over the transducer when the sheath is in place on the probe shaft; and
attaching the needle advancement assembly to the probe shaft where the sheath is positioned between the needle advancement assembly and the probe shaft over at least the entire length of the probe shaft.

22. A method as in claim 21, wherein the distal portion of the sheath further conforms closely to one or more attachment features on the distal portion of the probe, wherein a shape of the one or more attachment features is assumed by the sheath and wherein a user aligns one or more attachment features on the needle advancement assembly with the one or more attachment features on the probe before attaching the needle advancement assembly to the probe shaft.

23. A method as in claim 21, wherein a proximal portion of the sheath remains loose over a proximal region of the probe shaft after the sheath is positioned over the probe shaft.

24. A method as in claim 22, wherein the sheath allows visual indication of the position of the attachment feature on the probe, without visibility of the attachment feature.
